(19) United States
(12) Patent Application Publication
(10) Pub. No.: US 2024/0112774 A1
(43) Pub. Date: Apr. 4, 2024

(54) TOKEN MISALIGNMENT DETECTION AND REMEDIATION DEVICE

(71) Applicant: LogicMark, Inc., Louisville, KY (US)

(72) Inventors: Chia-Lin SIMMONS, Louisville, KY (US); Rafael SAAVEDRA, Louisville, KY (US); Peter WILLIAMS, Louisville, KY (US)

(73) Assignee: LogicMark, Inc., Louisville, KY (US)

(21) Appl. No.: 18/484,192

(22) Filed: Oct. 10, 2023

Related U.S. Application Data
(63) Continuation-in-part of application No. 18/375,155, filed on Sep. 29, 2023.
(60) Provisional application No. 63/412,084, filed on Sep. 30, 2022.

(51) Int. Cl. G16H 10/60

(52) U.S. Cl. CPC G16H 10/60

(57) ABSTRACT

A system, apparatus, and method align tokens representing the state of a person under care with at least one stakeholder represented by further tokens. The system identifies incentives in multi-party interactions where such incentives are represented by specification value pairs and through the use of game theory, machine leaning, pattern identification and recognition and/or digital twins in any arrangement, misalignment of such specification value pairs may be identified and responded to in manner that can avoid and/or mitigate the impact of such misalignments on the interactions of those parties, including in pursuit of the reduction of fraud or other undesirable behaviors within at least one system.

### CROSS-REFERENCE TO RELATED APPLICATIONS

This application is a continuation-in-part of U.S. patent application Ser. No. 18/375,155, filed Sep. 29, 2023, which claims priority to U.S. Provisional Application No. 63/412,084, filed on Sep. 30, 2022, and the contents of which are incorporated herein by reference.

### BACKGROUND

### Field of the Disclosure

Aspects of the disclosure relate in general align tokens representing the state of a person under care with at least one stakeholder represented by further tokens.

### Description of the Related Art

Many situations involving wellness and care for a person can involve a range of incentives, by and for that person, the systems designed to support that person, the other people, institutions and other stakeholders involved with that person, that are at least in part a representation of the interests of those stakeholders.

For example, these incentives can include; financial, safety, security, stress, influence, compliance, time management, optimization, theft, PR/Branding, reputation, marketing, competitive advantage, deception, social advantage, information management (including hiding/restriction/information retention and the like), effort minimizations, perception, privacy, management and a range of behaviors, such as jealousy, envy and/or other human individual and/or collective behaviors.

In many circumstances these incentives can be misaligned such that there are systemic and/or individual rorts of these systems and relationships. Most all systems have some constraints and can be worked for unintended consequences and/or for the benefit of and by malicious and/or self-serving actors.

### SUMMARY

Embodiments include a system, device and method to align tokens representing the state of a person under care with at least one stakeholder represented by further tokens.

In one embodiment, the system includes a plurality of environmental sensors. The plurality of environmental sensors monitor a sequence of interactions of a person under care resulting in a detected data set, and provide a first token. The first token comprises the detected data set representing quiescent behaviors of the at least one stakeholder in an environment. Each of the behaviors is represented by a multi-dimensional feature set comprising at least one pattern forming part of a health care profile for the person under care. A fraud avoidance prediction system comprises a transceiver, a non-transitory computer-readable storage medium, and at least one hardware processing unit. The transceiver receives the first token from the plurality of environmental sensors. The non-transitory computer-readable storage medium stores a second token. The second token represents current state behaviors of the at least one stakeholder in the environment. The at least one hardware processing unit compares the first token and the second token, and at least in part uses game theory to align the first token and the second token.

The comparison of the first token and the second token may result in at least one notification to the at least one stakeholder.

The alignment of the first token and the second token may be via a consequent incentive for the at least one stakeholder.

The alignment of the first token and the second token may align configurations of the plurality of environmental sensors.

The game theory may be a cooperative and non-cooperative games, normal form and extensible form games, simultaneous and sequential move games, constant sum, zero sum and non-zero-sum games and symmetric and asymmetric games.

In another embodiment, a machine-learning enabled fraud-detection system aligns tokens representing the state of a person under care with at least one stakeholder represented by further tokens. A transceiver receives a detected data set from a plurality of environmental sensors. The detected data set represents quiescent behaviors of the at least one stakeholder in an environment. Each of the behaviors is represented by a multi-dimensional feature set forming part of a health care profile for the person under care. At least one machine-learning processing unit receives the detected data set from the transceiver and creates a first token comprising the detected data set. A non-transitory computer-readable storage medium stores a second token. The second token represents previous representing current state behaviors of the at least one stakeholder in the environment. The at least one hardware processing unit compares the first token and the second token, and uses at least in part game theory to align the first token and the second token.

The comparison of the first token and the second token may result in at least one notification to the at least one stakeholder.

The alignment of the first token and the second token is via a consequent incentive for the at least one stakeholder.

The alignment of the first token and the second token may align configurations of the plurality of environmental sensors.

The game theory may be a cooperative and non-cooperative games, normal form and extensible form games, simultaneous and sequential move games, constant sum, zero sum and non-zero-sum games and symmetric and asymmetric games.

In another embodiment, a non-transitory computer-readable storage medium is encoded with data and instructions. When read by a computer, the instructions cause the computer to receive, via a transceiver, a detected data set from a plurality of environmental sensors. The detected data set represents quiescent behaviors of the at least one stakeholder in an environment. Each of the behaviors is represented by a multi-dimensional feature set forming part of a health care profile for the person under care. At least one machine-learning processing unit receives the detected data set from the transceiver and creates a first token comprising the detected data set. A non-transitory computer-readable storage medium stores a second token. The second token represents previous representing current state behaviors of the at least one stakeholder in the environment. The at least one hardware processing unit compares the first token and the second token, and uses at least in part game theory to align the first token and the second token.

The comparison of the first token and the second token may result in at least one notification to the at least one stakeholder.

The alignment of the first token and the second token is via a consequent incentive for the at least one stakeholder.

The alignment of the first token and the second token may align configurations of the plurality of environmental sensors.

The game theory may be a cooperative and non-cooperative games, normal form and extensible form games, simultaneous and sequential move games, constant sum, zero sum and non-zero-sum games and symmetric and asymmetric games.

### BRIEF DESCRIPTION OF THE DRAWINGS

To better understand the nature and advantages of the present disclosure, reference should be made to the following description and the accompanying figures. It is to be understood, however, that each of the figures is provided for the purpose of illustration only and is not intended as a definition of the limits of the scope of the present disclosure. Also, as a general rule, and unless it is evident to the contrary from the description, where elements in different figures use identical reference numbers, the elements are generally either identical or at least similar in function or purpose.

FIG. 1 is a block diagram of care village systems.

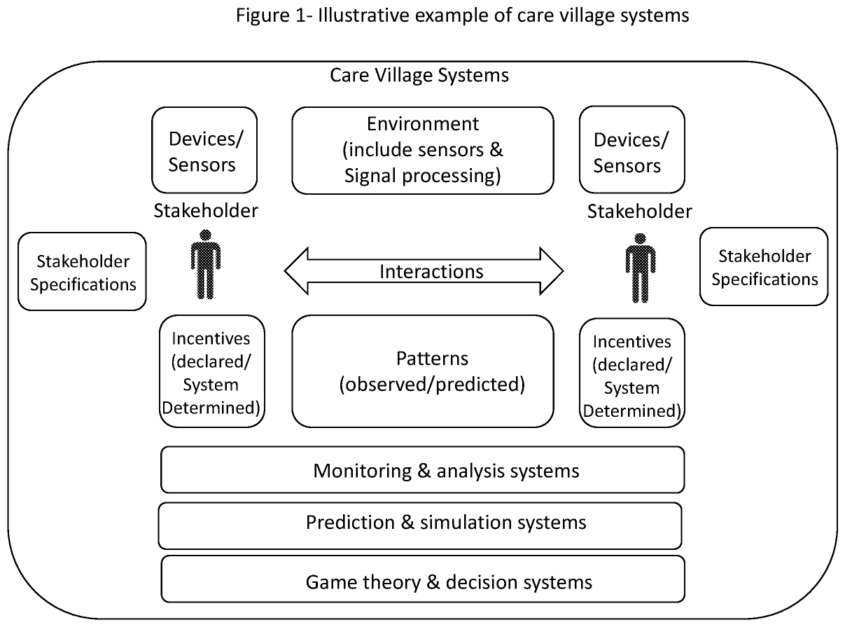

FIG. 2 is an illustrative example of fraud avoidance prediction systems (FAPS).

FIG. 3 is an illustrative example of FAPS, Care Village Digital Twins and predictive systems.

FIG. 4 is a further illustrative example of care village systems.

FIG. 5 is a further illustrative example of care village systems.

FIG. 6 is an illustrative example of operating CVDT.

### DETAILED DESCRIPTION

Aspects of the present disclosure include a system, device and/or method described herein that uses game theory and machine learning to identify and develop representations of potential and/or probable incentive misalignments within systems and/or with stakeholders of those systems. This can include incentive misalignments between sets of parties, within the system based on their relationships, both as individual actors and/or acting as a group. These representations may be defined as a set of systemic, collective and/or individual rorts of such a system. This use of game theory and machine learning, potentially in conjunction with digital twins, to identify “loopholes” of and/or in processes, operations, patterns, stakeholder actions and/or other care village operations involving stakeholders as participants, individually and/or collectively (for example in collusion), provides a unique approach to the effective, transparent and trustworthy operation of these systems, including those deployed in a care village.

One aspect of the use of game theory and machine learning in combination, at least in part, with one or more digital twins, is the identification of the balance of incentives that represents multi-stakeholder interactions. This can include ascertaining the balance of incentives that are beneficial or disadvantageous to one and/or a group of stakeholders. In some embodiments described herein, this is described as a Fraud Avoidance and Prediction System (FAPS)

The use of FAPS to identify interfaces and/or friction points and/or process bottlenecks and/or other impediments and the like, between stakeholders and/or the systems they interact with, can support the identification of sets of incentives that are indicative of manipulation, rorts, non-compliance and/or other misalignments.

The use of FAPS to provide prediction, avoidance and in conjunction with care village response systems, recovery from misalignment of incentives can provide care village systems and the stakeholders thereof with a transparent, trustworthy, robust and reliable operating environment.

A further perspective on the representation of the operations of the care village is the identification of value and the monitoring of the care village to establish metrics for value of provision of products and/or services from multiple stakeholder perspectives.

Value can be expressed in a number of metric terms such as monetary, health, wellness, stress, convenience, satisfaction and the like and can be considered, recognized and/or perceived in different terms by the stakeholders involved. Many of the current metrics used as transactional representations, may be inadequate to represent the state and operations of the care village and the stakeholders thereof, when considered from the perspective of wellness and care of such stakeholders.

For example, the quality of life for a PUM, and potentially other stakeholders can not necessarily be represented through transactional metrics, such as financial, procedure based or other quantization's expressed in transactional terms. For example, in a shared economy model such transactional value representations may be inadequate to express quality for such factors as reviews, evaluations, reputations and the like.

The use of a multi-incentive model for the representation of value can provide a more reflexive representation of the differing priorities, optimizations and/or perspectives of each stakeholder including through their interactions, interfaces and/or touch points as each may have differing internal and external incentives.

One of the aspects where there is an incentive for information manipulation and other fraudulent behavior is in reviews, evaluations and/or scores and/or the like, which can determine reputation indicators of care village stakeholders such as product and/or service providers, and may translate directly or indirectly into business opportunities and monetary income for the stakeholder. Prevention of such fraudulent behaviors can be achieved by using FAPS to identify process loopholes, also by using secure and easy to track transaction data recording methods, such as distributed ledgers, smart contracts and by using machine learning methods, including in combination with game theory, to detect fraudulent patterns in real, predicted and/or simulated scenarios.

In some embodiments, a stakeholder, such as a PUM, family, friend or neighbor may have or share a dashboard representation of the state of the stakeholder in regard to the particular events, service and/or products that such a stakeholder may interact with. In common with many other similar dashboards, this can include alerts, messages, confirmations, metrics, recordation's and other data that are pertinent to the stakeholders.

In some embodiments, metrics may be represented by tokens, such as NFT's. For example, the use of at least one distributed ledger for recording and tracking transactions and Care Village stakeholder reputation indicators allows for those indicators to be represented as tokens for automatic validation and/or for possible value exchange.

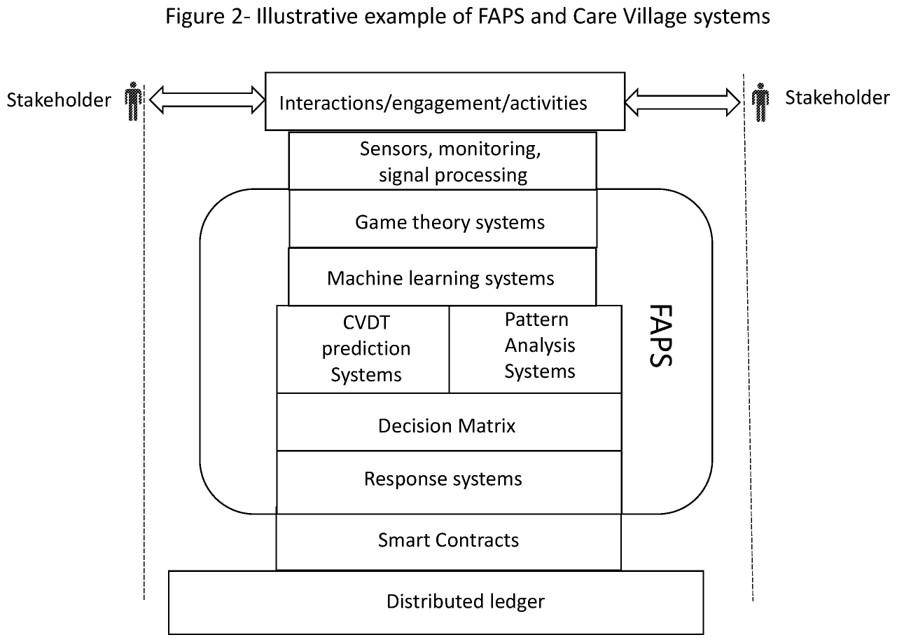

FIG. 2 illustrates an embodiment of a FAPS. In this example two stakeholders (101 and 102), have a set of interactions, engagements and/or activities (109), though in any real deployment, there can be a plethora of stakeholders having many interactions, engagements, activities and the like. Figure two shows only these two for simplicity. The stakeholders (101,102) are monitored, in whole or in part, by one or more sensors, devices (including those worn and/or carried), monitoring systems and/or care processing systems in any arrangement (202). The data sets generated by and/or the configuration and/or operations of such sensors, devices, monitoring and/or care processing (202) can be integrated, in whole or in part, with a Fraud Avoidance and Protection System (FAPS), illustrated here as 201.

Such a FAPS may include one or more game management systems (203), which incorporates one or more game theory-based games, strategies, payoffs and players thereof. Such game management systems may interact with one or machine learning systems, where such systems may be employed to evaluate one or more games, assist, in whole or in part in the selection and/or deployment of one or more games, evaluate one or more strategies, payoffs and/or player actions within such games and/or operate as a player, for example as a proxy for a stakeholder, in collaboration with a stakeholder and/or as an independent actor within one or more games (204). The operations of game management systems (203) and/or machine learning systems (204) may be complimented by the use of one or more Care Village Digital Twins (CVDT) and any predictive systems employing such CVDT (205). The configuration and/or operations of 203,204 and/or 205 may employ one or more pattern and/or token analysis systems (206) in any arrangement.

The outcomes of the configurations and/or operations of 202,203,204, 205 and/or 206 in relation to the interactions (109) of the multiple stakeholders (101,102) can be represented in a decision matrix (207), which may interact with one or more response systems (208). These outcomes, decisions and/or responses can be part of one or more smart contracts (209) and may be stored in one or more distributed ledger (210).

Patterns and Analytics

A care village comprises sets of stakeholders with various relationships, with at least one of those stakeholders being a person under monitoring (PUM), who has a health condition profile (HCP) and an environment in which they are predominately domiciled.

The care village operations are predominately based on patterns of behaviors, actions, events and/or other identifiable characteristics. These patterns provide the ability to monitor an individual, a PUM, in a manner that protects their privacy whilst enabling the necessary support to manage their HCP.

The expression and/or calculation of incentives as specifications and/or metrics which can be evaluated enables formulation of multi variate representations of one or more stakeholder's perspective on or about a particular set of circumstances, including any relationships with other stakeholders and/or the products or services they provide, for example those expressed as one or more patterns, which may be represented by one or more tokens.

There may be some incentives which are core to the stakeholder and will apply in most all circumstances, and others that may vary according to circumstances. Core incentives are those that are consistent across multiple times, situations and/or behaviors, including those represented by one or more patterns, whereas other incentives may be contextual to a specific situation, which may be represented by a further pattern. For example, a core incentive may be that a PUM prefers to avoid using premium or expensive products and/or services. In this example there may be two core incentives, the first being their preference for less expensive products and/or services and the second core incentive of a preference to evaluate multiple products and/or services so as to select an option that satisfies the first core incentive. These combinations of incentives can provide a framework for the evaluation of a stakeholder's intentions, actions and/or outcomes. The use, for example, of predictive systems employing, for example, machine learning techniques and potential embodiment as digital twins can enable prediction of such intentions, actions and/or outcomes so as to be potentially beneficial to the at least one stakeholder and/or the care village system as a whole.

In some embodiments one or more type of feedback may be used to establish, validate, confirm or otherwise identify the intentions of a stakeholder. This can include the use of games, where the stakeholder is presented with a set of alternative responses, which may, in whole or in part include their stated incentives, in some cases represented by one or more metrics, and related payoffs. For example, the payoffs may have tangible or intangible benefits to that stakeholder, and as such can provide further indications as to the accuracy of their stated intentions.

In some embodiments, incentives may be explicitly stated by one or more stakeholders, for example represented as specifications. Incentives may also be determined, at least in part, by the actions of a stakeholder in a set of circumstances, for example a behavior, represented by a pattern, such as eating, sleeping, exercise and the like. In this manner a stated or specified incentive may have a second order function which is the observed, for example by the one or more sensors, devices and/or systems, behavior where such observations are, at least in part differing from the stated and specified incentives.

Figure one illustrates an embodiment of the care village systems where multiple stakeholders, illustrated for simplicity here as Stakeholder S1 (101) and stakeholder S2 (102) have a set of interactions (109) within the care village (108). Such stakeholders may have expressed and/or have imputed, derived, predicted or in other manners incentives determined by one or more care village systems (110 and 111). Further each stakeholder has a set of specifications (103 and 104), which may include their HCP. Each of the stakeholders may be predominately domiciled in an environment (107) which includes one or more sensors and/or care processing systems. Further sensors can be deployed, such as those worn and/or carried by the stakeholders (105 and 106), including those embedded within one or more devices. These devices, sensors and systems can produce data sets that are represented by one or more patterns, which are observed during the interactions of the stakeholders with the environment and/or each other in any arrangement. In some embodiments this can include the use of predictive systems to predict such patterns representing behaviors of those interactions (112). Such prediction and simulation may be undertaken, for example, by one or more prediction and simulation systems (114), which may interoperate with monitoring and analysis systems (113), which may operate on the data sets produced by 105,106 and/or 107, incentives (111/112), specifications (103/104) and/or patterns (112). The game management and decision systems can interoperate, in any arrangement with the care village systems, for example data sets form 105,106 and/or 107, incentives and specifications (103/104/110/111), patterns (112), prediction and simulation systems (114) and/or monitoring systems (114).

In some embodiments stakeholders may be asked to rate their incentives, using for example a scalar representation. This may then be used as part of their initial framework to establish their intended behaviors as represented by the declared incentive ratings.

For example, this approach can involve simple scalars, such as 1 to 10, or may involve choices, such as rating incentive 1 vs incentive 2. For example, a slider with finance and stress management as the two variables. In this example the stakeholder can set a relative weighting for these incentives. Such scalars may, in some embodiments, be specific to particular behavior patterns, environments and/or stakeholder interactions. These interactions may be represented as sets of patterns, and may form behavior tokens. The use of one or more games, based on game theory may be employed to establish and/or determine these scalars. In some embodiments, the stakeholder may act as a player in such game as may an AI/ML, module.

These initial scalars may then be compared to the actual, and for example using machine learning represented in the form of digital twins (Care Village Digital Twins-CVDT), predicted behaviors. In some embodiments games and game theory may be used to, at least in part, determine such predicted behaviors. As Stakeholder actions unfold, represented, at least in part, by the patterns of their behaviors, which may be represented by one or more behavior tokens, the relationship between their declared and/or specified incentives and their actions becomes apparent and can be incorporated into one or more CVDT, to more accurately predict their behaviors in various circumstances. One aspect of this, for example, is behavior in regards to the financial impact of care, where for example a person may wish to minimize the costs and accept that their care will be less than optimum due to this.

Based on the operations of the digital twins the sets of incentives may be expanded as the observed behavior indicates further incentive structures, for example those operating across multiple stakeholders. The identification of such incentives may be undertaken, at least in part, by at least one machine learning technique.

In some embodiments, one or more games may be deployed, for example in one or more digital twins to determine, at least in part, the potential behaviors of one or more stakeholders, including multiple stakeholders. In this manner a probability of the behaviors may be determined by employing multiple games, which can inform one or more sensors, devices and/or systems as to the probable predicted behaviors of the one or more stakeholders. Such an approach can identify those behaviors of multiple stakeholders which can have a wellness and care, financial or other impact on the one or more stakeholders.

Such use of one or more games, for example repeated games, information transfer games, multistage games, risk assessment games and/or single games played in multiple digital twins any of which may have one or more ML/AI modules acting as player in such games, can lead to the determination and identification of strategies employed by such stakeholders and consequent appropriate incentives for one or more stakeholders that enable the multi stakeholder actions to result in a beneficial outcome.

In some embodiments a pattern specification language may be deployed for the creation and management of patterns used within care village. This may be based on pattern libraries that include pattern segments that can be constructed into sets of patterns. For example, a set of event sequences representing data received from at least one sensor, device and/or system may be instantiated as a pattern segment, with specifications for the sensor data, time periods, sensor configurations, location data and the like. Such event sequences may be managed by, for example a care processing system and may form part of at least one pattern. There are many available choices of computer languages in which such specifications may be written.

These patterns may individually and/or in any arrangement form behavior tokens, named bevokens, that can represent, for example Time of Day (ToD) and/or quiescent or event behaviors represented as tokens.

An aspect of the system is the representation of events, actions and other activities as sets in the form of patterns, where in some embodiments these patterns may, in whole or in part, form one or more behavior tokens, described herein as bevokens. This approach mitigates the need to evaluate each individual sensor, device and/or system generated event, action and/or activity individually as it is the relationship between the events, actions and/or other data sets that form the patterns that are evaluated. This approach focuses on both the predictive and responsive system operations, such that if a pattern and/or behavior token exhibits sufficient deviation from the expected, specified and/or previously established parameters, then the appropriate response mechanisms may be invoked. This operating granularity at the level of patterns and/or tokens and sets thereof provides an efficient and effective monitoring approach, that can support the privacy of one or more stakeholders, that inherently enables the detection of misaligned incentives, disruptive activities, dishonesty and/or other behaviors that can affect the operations of the care village.

For example, patterns may be representations of the state of a PUM and their environment, including for example, any other stakeholders. In some embodiments each state may be represented, in whole or in part, by one or more patterns.

The use of pattern and/or token evaluation techniques enables the consideration of those patterns and/or tokens from the perspective of multiple stakeholders. This can identify potential discontinuities and incentive misalignments that may form part of a set of interactions between the stakeholders. Such evaluations may then be used, for example in digital twins with the benefits of machine learning, to evaluate alternative interactions, from the multiple perspectives of the differing stakeholders, so as to identify potential optimizations to those interactions. Such an approach may also be used to evaluate the relative incentives that one or more stakeholders may have in a set of interactions, and identify any behaviors that are disadvantageous to another stakeholder.

One aspect of the system is the use of patterns and/or tokens for multiple stakeholders, where those patterns and/or tokens may be, for example, based on templates, and represent common behavior sets for the stakeholders, though the outcomes for each of the stakeholders may differ. These differing outcomes may have dependencies on inputs, environment and/or outputs within the same pattern framework.

The use of patterns to identify, discover, predict and represent any frictions, tensions and/or discontinuity in outcomes and/or responses for multiple parties represented by these patterns and/or tokens through their interactions can provide a systemic approach to the avoidance or mitigation of such situations. Where there is an inevitable interaction where the incentives of the parties are not well aligned, for example on the pricing of a product or service, this discontinuity in alignment may form a focal point for identification of activities that may have a high probability for at least one party to manipulate or rort the situation. This may be the case where one party takes advantage of another, for example where one party has a health condition in which their mental faculties are deteriorating, and another party attempts to exploit that situation for their own benefit. This can include the identification of pattern and/or token interactions that represent predatory behavior characteristics.

One aspect for use in the evaluation of patterns and/or tokens is the assessment of the outcomes and/or responses. For example, if a pattern and/or token has an outcome or response where each of the parties involved with the pattern and/or token has no or negligible loss or gain, as determined by the parameters of the pattern and/or token, then the outcome or response can be determined to be neutral. In some embodiments this may be assessed, at least in part through one or more games, where the parties involved are players. In the example where at least one party has benefit at the expense of another party, such an outcome or response can be evaluated in regard to the specified incentives for each party to ascertain whether that outcome or response represents the intention of one party to manipulate or rort the system for their own benefit, often to the detriment of another party, such as one involved in the game.

Within these outcome and response measurements, there may be representations of the degrees of alignment of at least one party with their expectations and predictions for those outcomes. These degrees of alignment may be expressed as metrics, including those for satisfaction, wellness and care, stress and the like and can include integration of feedback, provided by at least one party, though generally such feedback can come from multiple parties, including the system.

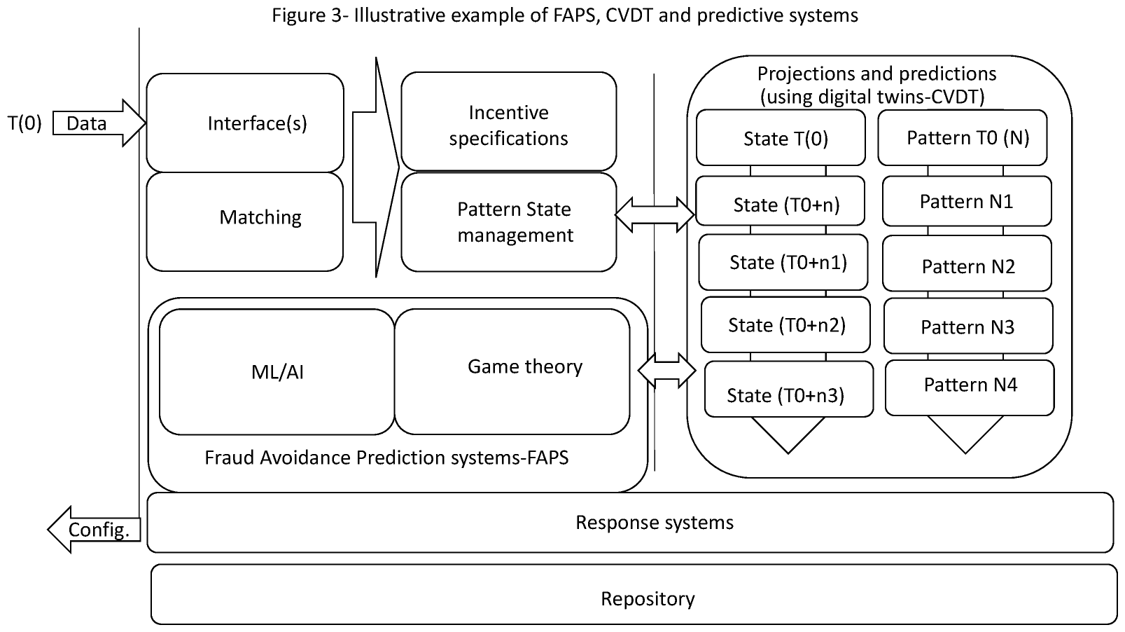

FIG. 3 illustrates an example deployment of a FAPS which includes one or more CVDT and predictive systems. In this example, data is input from one or more sensors, devices and/or systems involved in monitoring a PUM in an environment (301). This data is ingested by one or more interfaces (302), where for example, one or more matching systems may be employed (303), to in whole or in part, match this data to one or more patterns, such as those managed by one or more pattern state management systems (305). These patterns and/or data sets may be compared to and evaluated in light of a set of incentive specifications (304), where those specifications, which can be either explicit or implicit, are correlated, at least in part with the patterns of the pattern state management (305). One or more CVDT (306) may use a set of states for each of the patterns, for example a pattern (P1) may be present at a time (t0) and the CVDT prediction systems may iterate that pattern as a representation of a set of state changes over a period of time, for example represented by increments of n (n1, n2, n3), which are represented by corresponding patterns, such as N1, N2,N3,N4. In this manner the CVDT and predictive systems may provide foresight of the potential changes in state and the patterns corresponding to those state changes that the PUM may experience.

These predicted state changes and corresponding patterns may be communicated to a game management system (203), as may the stakeholder incentive specifications (304), where games representing, in whole or in part, theses states and/or patterns and their predicted changes may be evaluated in terms of one or more games representing these states, patterns and/or variations. The operations of the game management systems (203), CVDT and predictions (306) and/or the matching systems (303) may be integrated with a machine learning/artificial intelligence module (307) so as to apply the one or more appropriate machine learning techniques in any arrangement. In some embodiments, game theory systems (203) and a machine learning/artificial intelligence module may be part of a FAPS (201) embodiment.

The outcomes and/or results of these evaluations and/or processing may be communicated to one or more response management systems (309) and/or a repository (310), which may also receive response outcomes. In some embodiments, the response management systems may communicate one or more responses, for example as actions (311), such as configuration data sets, specifications and/or instructions, data or other actions to one or more PUM, sensors, devices and/or systems and/or one or more stakeholders in any arrangement.

Such an approach can provide the capability to identify optimum sequences of multi-party outcomes, reporting, feedback and/or validations or other metrics. In this manner the types of patterns and their outcomes, as well as the sequence sets comprising those patterns that indicate a detrimental activity to at least one stakeholder can be identified. Such identification can provide and enable the degree of compliance or non-compliance, such that the degree of variations may be assessed and where required remedial or other actions can be undertaken.

Part of this approach can lead to the discovery, including through the use of machine learning techniques, of stakeholder patterns, where the behavior of a stakeholder is identified. In the example where there is a pattern that represents a stakeholder repeated behavior to gain advantage or rort the system at the expense of another stakeholder and/or the system itself, for example through non-compliance with the systems specifications, such a stakeholder may have their access to one or more system, stakeholder or other care village entities restricted and/or limited.

These stakeholder patterns and/or tokens can provide an effective approach to determining those collaborations between multiple parties that rort, disrupt and/or create incentive misalignments through the actions of these multiple stakeholders. For example, if a provider of a product and/or service, in collaboration with a receiver of a product and/or service, collaborate to charge another stakeholder with the cost of provision whilst not actually providing the product and/or service.

One aspect of the system is the use of incentive specifications, in combination with, for example game theory and/or machine learning to enable the prediction of misalignments of incentives and/or deviations of behavior that can lead to stakeholders attempting to avoid compliance with at least one set of specifications.

The use of one or more games to represent the incentives of the stakeholders supports the identification of those strategies employed by such stakeholders within the games that represent their incentives. For example, a game where there are alternate outcomes, for example financial versus health, can be used to establish the relative weightings of those incentives, for one or more stakeholders in a set of circumstances. Using varying payoffs can further accentuate or diminish the variations in the weightings that represent the stakeholders' incentives. This approach may include multiple games with different strategies that can, in some embodiments, be played by one or more machine learning module acting as a proxy for one or more stakeholders. When combined with digital twins, such games, strategies, payoffs and/or outcomes may be evaluated so as to determine, at least in part the incentives of one or more stakeholders, including those that have not been explicitly stated by such stakeholders.

The evaluation of variations in behaviors, particularly when such variations create a pattern, which may in whole or in part be represented by one or more token, supports the identification of at least one intent of such variations and/or deviations that are likely focused on fraud and/or compliance avoidance. The use of sensors, devices and/or systems to monitor the activity, in relation to the operating pattern and/or token of one or more stakeholders within the context of an HCP can provide an initial alert and/or trigger for configuration of that and/or other sensors, devices and/or systems to verify such variation and deviation. The degree to which this occurs may cause at least one system response and/or may invoke at least one process for monitoring of such variations and/or deviations over a period of time, for the purpose of detecting a pattern representing a change in behavior of a stakeholder in relation of the system and/or another stakeholder.

In some embodiments a stakeholder may determine the terms of their service, expressed for example as specifications forming a smart contract. These contracts may be evaluated to establish the incentives underlying these specifications which may then be incorporated into the monitoring systems. Within such smart contracts, the care village systems may operate so as to effect limitation of variation and/or deviation opportunities.

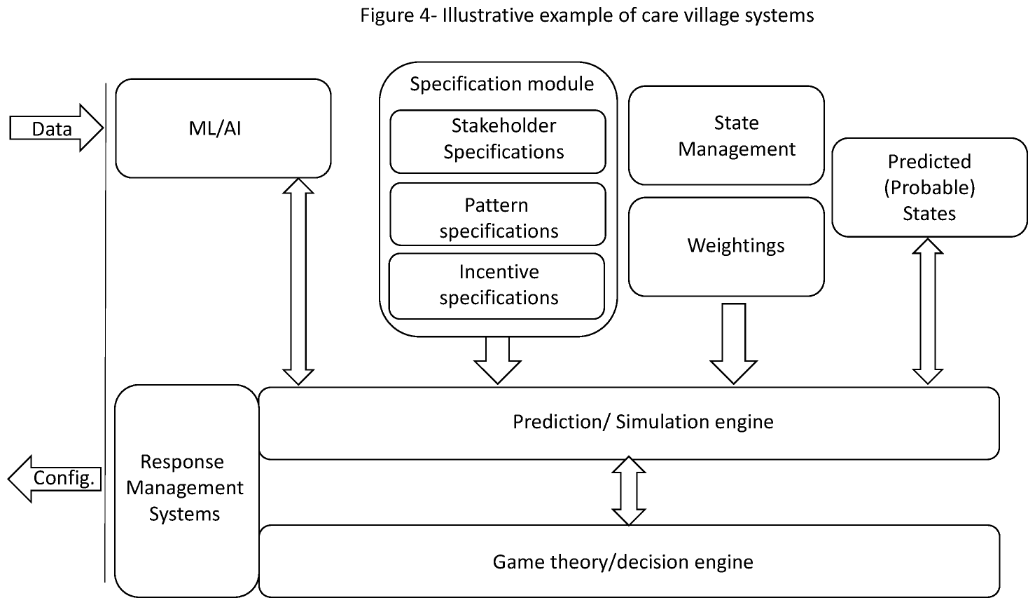

FIG. 4 illustrates an example of care village systems that include predictive systems. For example, sets of data (401), such as that generated by one or more sensor, device and/or system that is involved in monitoring one or more PUM and/or other stakeholder in one or more environments may be ingested into a machine learning/artificial intelligence module (307). In this example, one or more CVDT (409) may be employed with predictive systems, which may include one or more machine learning techniques embodied in a machine learning/artificial intelligence module (307), to create one or more simulations of the behaviors, state, patterns, interactions, incentives, specifications, compliance and/or other characteristics of those under monitoring. These predictive systems and/or simulations may operate on and/or with one or more sets of specifications that are managed by, for example, a specification module (402). In this example such specifications may include, stakeholder specifications (403), pattern specification's (404) and/or incentive specifications (405), the latter being those of the one or more stakeholders. These specifications (402) and the incoming data sets (401) may be represented by one or more states, managed by the state management systems (406), which can have various weightings, parameters, priorities and/or other attributes that can be used by, for example the CVDT predictive and simulation systems for calculations and/or processing, which are managed by an attributes module (407). This can include sets of attributes from other similar PUM and/or stakeholders which are similar to those that the incoming data (401) represents. Such attributes may be derived from other care village operations and can be anonymized.

A CVDT predicted state management module (408) may retain the probable states for one or more situations, including those that commonly occur as well as those representing the most likely states, based at least in part on the data sets. These predictions may then form part of one or more games, managed by the game management systems (410), so as to generate one or more decision matrix informing at least in part, a decision engine (411). The outputs for this engine (411) may be communicated to one or more response management system (309), which may then instigate one or more actions (412) which may communicated to sensors, devices, systems and/or stakeholders in any arrangement.

Game Theory

In many circumstances the majority of operations within and by the care village sensors, devices and/or systems can be represented in the form of structured interactions, subject to at least one set of specifications, with the one or more stakeholders of the care village. These specifications can be, in some embodiments, characterized as games that involve the interactions of patterns that are operating as representations of the behaviors of the stakeholders within the care village. This can include interactions that are physically undertaken in real time and may involve one or more digital representations in the form of one or more digital twins. In some embodiments involving many circumstances, the alignments of incentives of the stakeholders can be represented as having an equilibrium in the sense of the use of game theory. Further the underlying dynamics of the game representing such incentives and the actions and events therefrom, can represent the expectations of the stakeholders and/or sensors, devices and/or systems engaged in such activities.

For example, if a set of incentives and/or interactions of a set of stakeholders within a care village can be represented as a finite non zero sum non cooperative game, then there is a Nash equilibrium, which involves mixed strategies of that set of stakeholders.

Although much work has been undertaken in applying game theory to economics, the application to a care village involving multiple stakeholders, with economic and/or health and wellness outcomes present a new and unique approach to avoidance, mitigation and/or reduction of rorts, fraud, or other detriment behaviors involving, financial and/or health and wellness-based misalignments.

In many stakeholder interactions the beliefs of one stakeholder regarding the actions and intentions of another stakeholder, for example a PUM and their medical doctor, will highly influence their incentives and actions in regard to those stakeholders. In this example, the game that could represent such a situation is a version of the ultimatum or dictator game, where the role of dictator is taken by the medical doctor. The exchange of value that is transacted in the game is the health of the PUM, including in some variants of the quality of life of that PUM. For example, a particular medication to treat one aspect of the PUM's health may have other effects that deteriorate their quality of life.

Within a care village the set of interactions is both finite and to a degree predictable, however the strategies deployed by the various stakeholders in pursuit of their own incentives may vary considerably.

One aspect of the system is the evaluation of the HCP and the patterns therein, which may be represented by one or more tokens, to establish which game, suitable for game theory, may be applicable. This can include establishing the incentives and events, including sequences thereof, within a pattern and/or a set of patterns, so as to evaluate the appropriate game, including one or more strategies, to represent those incentives, events, actions and/or outcomes of those patterns, which may include representation by one or more tokens.

In some embodiments, this can include a matching system whereby a set of games is held in a repository and the matching system evaluates the HCP and the patterns therein, both operating, previous and/or predicted, so as to determine which game(s) are the appropriate representation of those patterns, which may be represented by tokens. This can include multiple games representing a single set of patterns and/or tokens, where for example these games operate within one or more digital twins and the outcomes of these games are matched to the real-world activities in pursuit of establishing the best match. These matches may then be recorded and used in further matching operations, such that, for example a pattern and/or token or set thereof may have an associated game that is deployed when certain criteria are met, such as a type, class and/or other classification of one or more stakeholders, HCP, pattern sequences, set of tokens and/or the like. In some embodiments, machine learning may be applied in support of such matching systems, for example, to identify appropriate classifications, matching criteria and/or the like.

In the situation where an existing game does not achieve a sufficient match with the pattern and/or token or set thereof under evaluation, the system may deploy elements of a game, such as a payout matrix, relative risk/reward values and/or weightings, whether the game is zero sum or non-zero sum, types of strategies deployed by the stakeholders, decision matrix types and the like, to establish the framework for a new game, whereby this game may then be deployed and stored in one or more repository.

These new game frameworks may then be compared with the existing games held in the repository and where applicable modified or adjusted to conform with those games. As the interactions between stakeholders are generally well understood within the care village, most situations will likely be addressed by this approach. However, if a new game is identified, this will be added to the game repository. Such an approach creates an adaptive approach to identification of new potential games and strategies therein that are likely to evolve as the operations of the care village and the stakeholders therein operate over time.

A further aspect is the use of machine learning and digital twins to evaluate potential games, in part to identify the validity within the game of one or more equilibria. This can involve a machine learning module representing a player, for example a stakeholder, where the potential strategies of the stakeholder may be deployed, each with differing incentives. For example, if the incentive is to defraud another stakeholder, for example through charging an insurance company for a product or service that is not supplied, then this is undertaken in one or more digital twins to identify, at least in part the appropriate game to identify such an incentive and the applicable strategy that is applied by the stakeholder to achieve this outcome. In this manner the system may identify the actions of one or more stakeholders as being misaligned with the care village systems. The same approach may be used to identify other behaviors, such as altruism, tit for tat, exchange of value and the like of the one or more stakeholders involved.

In some embodiments, much of this evaluation by the matching system can be undertaken on a best fit approach, though, for example, fuzzy logic and/or other probabilistic, combinatorial, and/or other statistical methods, including the deployment of machine learning techniques.

Many of these games may be used in the training of at least one machine learning technique, such that both an existing corpus may be used for machine learning training and/or the games may be used to create such a corpus.

One aspect of the use of games and game theory is the alignment of the incentives of, and for, stakeholders with the parameters, weightings, risks, reward and decision matrix elements of the game

Such games may be deployed across sensors, devices, systems and/or infrastructure so as to incorporate one or more games where such games may inform the operations of such sensors, devices, systems and/or infrastructure, though for example an appropriate decision matrix, one or more outcomes, one or more strategies and/or one or more equilibriums. In some of these deployments, the decision matrix may be expressed as the outcome, and as such the sensors, devices, systems and/or infrastructure may adopt differing strategies to achieve, in whole or in part those outcomes. For example, this may include the configuration of such sensors, devices, systems and/or infrastructure, where the outcome of such a deployed game is the configuration of a set of sensors and/or other devices that have a relationship with those sensors, devices, infrastructure and/or systems that are operating such a game. For example, a set of sensors with a care processing system may deploy a specific combination of sensor data and algorithmic processing to create a data set that complies, at least in part, with such a decision matrix.

Such an approach may include at one or more communication devices which can provide a message to one or more stakeholders, such that the decision matrix for a specific game in which the communication device is a participant, can be affected by an action, decision and/or message from a stakeholder. For example, a stakeholder may receive a communication from a set of sensors, devices and/or systems that indicates the likelihood of a detrimental wellness event occurring to a PUM, and that stakeholder may then send a message to, for example, configure a set of sensors to monitor such an event, alert another stakeholder and the like

In another example, when a potential misalignment of incentives is represented by a game outcome, for example in a decision matrix. For example, this state can be communicated to a stakeholder, potentially with one or more messages prompting or suggesting one or more resolutions, such as for example an action, of such imbalance, so that stakeholder, including through their proxy, can take an action in response to such imbalance.

In some circumstances a game may be deployed in a digital twin, representing the unfolding events of one or more stakeholders, including potentially sensing and care processing sensors, devices and/or systems, such that if these unfolding events cease to match the current game parameters, including the strategies deployed by one or more stakeholders and/or sensors, devices and/or systems, then this can indicate that at least one of the stakeholders, sensors, devices, systems, care processing or other system elements is not conforming to specifications and/or has deployed a strategy that has not been previously identified. This can indicate that such a situation represents a malicious or detrimental misalignment of incentives, such as when a stakeholder is rorting the system and/or a fault or error within the operations of the systems themselves.

In many cases these evaluations can be undertaken by one or more monitoring systems, including those that employ one or more digital twins. This can involve human intervention, machine learning module assistance and/or further monitoring where appropriate.

In some embodiments the game creation, matching and evaluation systems may include:
Parameters for a running game
Establishing running game as part of a hierarchy
Informing a choice to switch to another game
Informing as to invoking further games

Game Systems

In some embodiments there can be a game management system that can operate in conjunction with the other system modules to determine the appropriate games to be deployed and the relevant hierarchies and/or selection criteria of such games. This can encompass both the operating games deployed in real time and those operating in digital twins to evaluate both past and future operations. This can also include the selections to be deployed for one or more machine learning techniques, for training and/or operational deployment.

In some embodiments a sensor, device and/or system can have at least one game management system and/or components thereof, which can be integrated into such a sensor, device and/or system and/or be accessible to that sensor, device and/or system through one or more communication networks. The care village system infrastructure components can have access to one or more game management systems and/or components thereof and/or one or more set of games. For example, a care processing system may have access to sets of games to determine, at least in part, which strategies are being employed by the stakeholders in a situation and can determine which care processing elements best match those circumstances.

In some embodiments, game systems can be provisioned so as to support collaboration with and among underlying operating environments of one or more sensors, devices and/or systems. For example, games that are cooperative can be employed to align configurations of one or more sensors, devices and/or systems with other sensors, devices and/or systems and/or care processing and/or systems and/or infrastructure so as to provide additional data as to a potential, predicted, actual and/or previous misalignment of incentives. For example, if a game operating on a digital twin representing an interaction of a stakeholder with another produces an outcome that suggests a misalignment, through for example the outcome of a game, another sensor, device and/or system may be invoked with a collaborative game to ascertain whether such a misalignment is occurring.

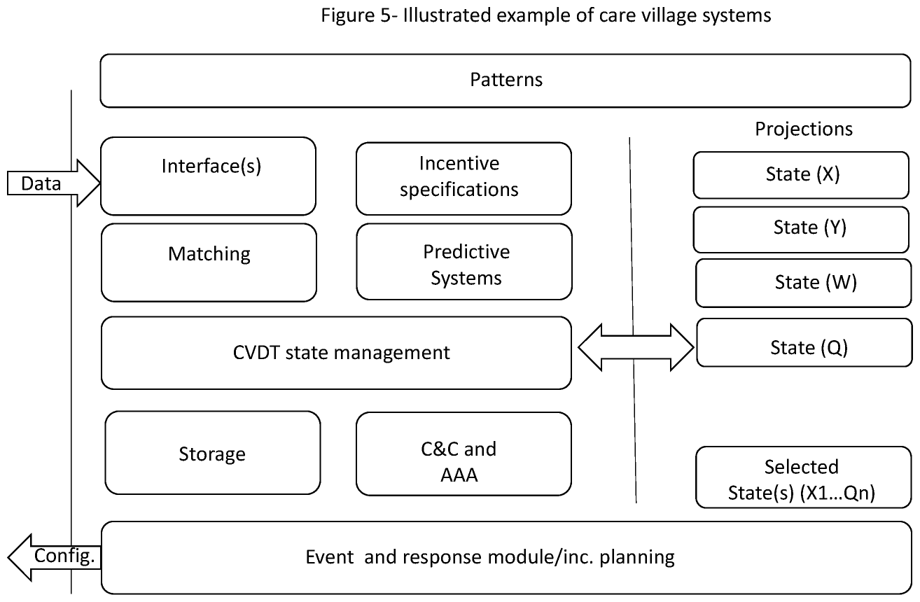

FIG. 5 illustrates an example embodiment of the game management systems (410) which in conjunction with one or more machine learning modules (505) and/or one or more CVDT (507) in combination with game identification systems (502) may process data sets received form the interactions (505) of the one or more stakeholders (504), which can be represented by one or more bevokens and/or patterns (503) and may include one or more incentive specifications (506). The game management systems (410) may use one or more repository (501) for the storage and management of games, strategies, frameworks, tokens (including bevokens), incentive specifications, stakeholder specification's, patterns, players and/or payoffs and any other pertinent data in any arrangement.

In some embodiments, game management systems and the games thereof, may differ for machine to machine, for example device to device, sensor to sensor and the like interactions, than those employed for machine to human, for example involving a human stakeholder, and human to human interactions.

The range of games employed in the machine-to-machine realm, may include cooperative and non-cooperative games, the outcomes of which may be used to evaluate patterns, including tokens, actions and/or events of a set of stakeholders for the assessment of any incentive misalignments.

Each type of game may be deployed in any arrangement for the differing circumstances and interactions between stakeholders, systems, devices, sensors and/or other care village entities. These include, for example, cooperative and non-cooperative games, normal form and extensible form games, simultaneous and sequential move games, constant sum, zero sum and non-zero-sum games and symmetric and asymmetric games, which can be deployed in any arrangement. Sensor, device and/or system objectives can be determined at least in part by sensor, device and/or system configuration, controlled for example by a care processing system, and may be further impacted by both sensor, device and/or system purpose and/or further game specifications, including the deployment of differing games for differing circumstances, held for example in memory and/or in an available repository of such a sensor, device and/or system.

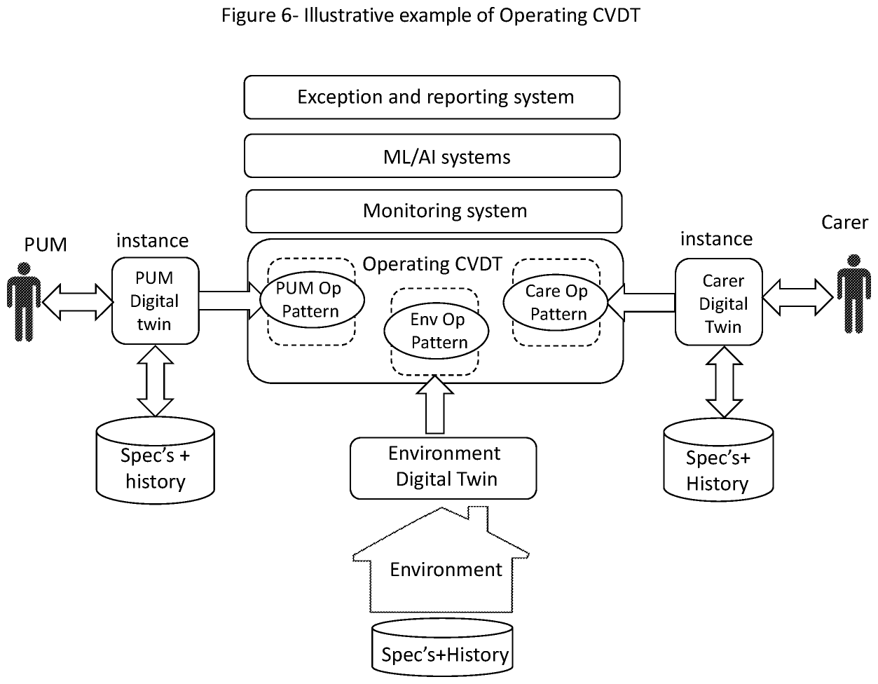

FIG. 6 Illustrates a set of operating games (605), where one or more stakeholders (607) are players within such games. These game players may include ML/AI modules operating as proxies for one or more stakeholder (601) and/or ML/SI modules operating as independent players (602). These players may use one or more bevokens, representing at least in part, one or more patterns (609) as part of the operating games. The interactions (606) between the stakeholders (607), their proxies (601) and any ML/AI players (602) may be, in whole or in part, influenced by stakeholder incentive specifications (608). These operating games may be instantiated and/or supported by the game management systems (613), including the game identification systems (612), which can evaluate the interactions (606) and/or the players (601,607,602), bevokens and/or patterns (609) and/or any specifications (608) to ascertain the appropriate games for operations. This evaluation and the game management systems operations may be supported by one or machine learning module (603), one or more CVDT and/or predictive systems (604), one or more repository (610) and/or one or more smart contracts/distributed ledger (611).

### EXAMPLES

One aspect of the system is the determination and representation of the range of potential payoffs and/or forms of decision matrix expressed as outcomes of a set of games. This can include the use of tokens. The mapping of a range of incentives related to and/or involved in a set of patterns, which can be represented by tokens, and their representation as a set of games that initially encompass the interactions of stakeholders within the intended and likely sets of interactions, can be formed into a repository. Although this repository will initially represent the common interactions within a care village, there may be unforeseen combinations, actions, events and/or consequences from and within these interactions, and as such a game management module supports an adaptive and extensible repository management system. This can include operations by both machine and humans where necessary. Such a repository may be distributed across the cloud, edge and other distributed systems.

The use of hierarchies of games can, for example, enable the care village systems to manage and invoke multiple games and game systems in support of the identification, avoidance and/or reduction of incentive misalignments and/or conflicts involving locations, time and/or resources. One aspect of this is the enablement of effective collaboration and cooperation between care village systems, stakeholders and/or sensors, devices and/or systems. For example, improving, optimizing and/or managing resource deployment, use and/or utility in pursuit of specifications, compliance and/or other objectives can be enhanced by such an approach.

In some situations, the relative relationships between care village stakeholders may be asymmetrical, in that one stakeholder has significantly more leverage than another. This can be represented in the incentives of each party, for example using a weighting schema, and can be further represented using asymmetric games and associated strategies. Asymmetric games may include, for example, princess bride games and the like, and have differing, typically opposite, incentives for each of the players. These differences can be accentuated by varying the payoffs for each player. This is particularly useful when applied to differing populations, for example providers of products and/or services and consumers or users of same. Such an approach can incorporate evolutionary dynamics, whereby the players use of mixed strategies may lead to an equilibria, which represents a stable state, for example a quiescent state represented by one or more token.

The use of evolutionary dynamics as a predictive method is well suited to the deployment of machine learning, where iterations of extensible form games can be undertaken with multiple probabilities for payoffs, for one or more players, and can indicate those strategies and/or situations where the incentive misalignments are likely to produce activities by one more stakeholders that rort the care village systems.

In some embodiments, an application of the stable matching problem may be incorporated as, at least in part, a technique to address the problem of stakeholders having differing incentives in and for their interactions. This approach can be deployed to create a matching of incentives, such that the predicted interactions can then be evaluated in light of the actualized interactions and any misalignment detected. Further this approach may be used to determine such potential misalignments, such that corrective actions may be taken in advance of the actualized interactions with the intention of reduction and/or potential removal of such misalignments. For example, if a stakeholder has incentive A with a value on N and another stakeholder with whom they will interact has a comparable incentive, including a direct equivalent, with a value that is a fraction of N, then by proposing to each stakeholder a differing values for those incentives, either through financial, time, effort or other characteristics, such misalignment may be averted, diffused or mitigated.

In some embodiments, incentive specifications may include metrics that relate individual incentives into sets, such as continuums, dimensions, number lines or other scalars. For example, a metric on a scale of 1-10 may be employed where the axis for that metric is financial cost at one extreme, and time of service provision at the other, where for example, finance is represented as 1 and time of service provision as 10, and for example this is conveyed to a stakeholder as a slider on a scalar, such that the stakeholder selects a value that represents the relative importance of the relative two incentives to each other. This can be extended to include numbers of incentives, each of which can be considered as, at least in part, a dimension for which stakeholders can select at least one value. These can form multi-dimensional metrics in any arrangement.

The relative values of incentives may be varied such that a stakeholder who ascribes a high value to one incentive may be persuaded to vary such a value in light of other incentives that another stakeholder may offer.

For example, a stakeholder may have a cost incentive which is orthogonal, in opposition, in conflict and/or in contradiction, to a quality-of-service incentive, where to avoid misalignment, the stakeholder and/or the service provider may, for that specific instance of the service provision need to adjust the values they have ascribed to these incentives. Such arrangements of values and incentives may then be stored, and in some embodiments, may become, at least in part, part of at least one smart contract.

The use of such an approach can yield a perceived fairness of the distribution of work, finance, effort, wellness outcomes and/or other determinates within the system, such that each of the stakeholders has less overall incentive to feel that they are being mistreated, disadvantaged or in other ways not benefiting from the care village operations.

Games may be deployed locally, based on device capability and/or remotely, depending on device communications in any arrangement.

Machine Learning

In some embodiments, a combination of machine learning and game theory for identification of patterns, the incentives of and for those patterns, potential and actualized strategies of the stakeholders in relation to each other and the patterns and discovery of other indicators and/or metrics can enable the prediction of potential situations that may require intervention and/or response by the system, another stakeholder, emergency services and/or other parties involved in the provision of wellness services, involving at least one decision management system to interact with such response and intervention systems.

In some cases, this may involve the deployment of configuration data to one or more sensors, devices and/or systems so as to more fully monitor a situation and, for example, alert a human response team and/or an automated response system.

For example, a GAN (Generative Adversarial Network) may be trained in line with the principles of game theory, in that the players, may be generative or discriminative, where each approach is applied to a corpus, represented in part by the sensor, device and/or system data and operating patterns, which may be represented by one or more tokens, with differing and yet complimentary objectives.

Another approach involves using multi-agent techniques, such as multiagent reinforcement learning or similar techniques, where each of the agents becomes a player in one or more games, developing strategies that improve their outcomes as they tend towards equilibrium.

Adversarial training of neural networks may also be used, where different adversarial techniques are applied and represented as players in one or more games with the intention of improving their strategies.

The use of game theory with machine learning involves the transformation of machine learning characteristics to those deployed in game theory. For example, if deploying a fair value to the various incentives for stakeholders, such as in Shapley and similar algorithms, each of the machine learning features, their ranking and range of target values may be correlated to, for example, players, strategies and outcomes.

One aspect of this is the relative marginal cost of, for example, a change to the value ascribed to an incentive, or set thereof by a stakeholder. The machine learning may operate on a wide or selected corpus including selections or combinations of, for example, incentives, patterns, sensor data, events, environmental attributes and/or other care village data sets. If stakeholders can converge on the ascribed value of their incentives, where those incentives are the same or sufficiently similar, the relative incentive for any one stakeholder to attempt to disadvantage another, rort the system or in other ways act in a manner counter to the care village operations, is significantly reduced. Further if a stakeholder continues to operate in such a manner as to take advantage of other stakeholders and/or care village systems in a systemic and continued manner, that stakeholder may have access to the care village reduced or revoked.

One aspect is the identification of those incentives that involve direct interactions between stakeholders, which in some embodiments may be instantiated as a graph, where for example those incentives, which can be considered as variables in this context, may directly interact and be represented by edges and those that indirectly interact, which may be considered as conditionally independent, such that they can depend on the values of other incentives.

In some embodiments, predicting incentive misalignment can employ one or more machine learning models. For example, in some embodiments, this process of predicting the likelihood of an incentive misalignment occurring at an early stage may involve the utilization of one or more machine learning models that are optimized to detect early variations form one or more patterns, including those represented by tokens, that represent the behaviors and/or interactions of one or more stakeholders. These models can be trained using extensive datasets that encompass a range of data sources. These sources can include sensor data, which can provide insights into environmental and contextual variables, as well as stakeholder behavior data. These datasets are curated to capture relevant patterns and correlations that may signify the emergence of incentive misalignments.

The training data is further enriched with instances of past incentive misalignments, enabling the machine learning models to discern critical patterns that indicate the presence of misalignments. Through a process of iterative learning, these models become adept at recognizing subtle indicators that might otherwise go unnoticed by human observers. By analyzing the interactions between sensor data, stakeholder behavior, and historical instances of misalignments, the machine learning models develop a sophisticated understanding of the potential risk factors associated with incentive misalignments.

This can be defined as the separation of each of these representations and may be part of determining the dependence relationships between incentives and/or sets thereof, in both directed and undirected representations. One aspect of the interactions of stakeholders is their declared and undeclared values for incentives. Not all stakeholders may declare all of their incentives or the values for them, and as such the system may calculate sets of incentives and values that can be ascribed to stakeholders in differing situations. In this case determination of the declared and undeclared relationships between incentives and their values may be represented by, for example, a graph, which can then be used to evaluate any incentive misalignments. This identification of interdependencies can be used to assist stakeholders in evaluating the tradeoffs, strategies, payoffs and any other consequences based, at least in part, on the alignment of incentives and any representations of those incentives in the context of an application of game theory.

In some embodiments, the system may provide a graphical representation of these variables within the framework of a game so as to enable a stakeholder to undertake operations involving these incentives and their values, for their benefit in a simplified manner.

In some embodiments feature extraction techniques employed by a machine learning technique may be configured to support machine to machine and machine to human communications.

One aspect of this approach is determining optimal actions to address any detected and/or predicted misalignment. For example, in some embodiments, when an incentive misalignment or the risk of its occurrence is identified, machine learning models can be used to determine suitable actions to mitigate or eliminate the incentive misalignment, its risk and/or its impact. Drawing from comprehensive datasets containing records of various incentive misalignments and the subsequent actions taken, these models can forecast the outcomes of different response strategies. The learning process involves understanding how particular actions interact with specific types of misalignments, and how these actions influence stakeholder behavior and system dynamics.

For example, the machine learning models can become proficient in recommending the most appropriate actions based on the unique context of each situation. This proactive decision-making approach can help swiftly address emerging misalignments, minimizing their negative repercussions, and fostering an environment of alignment and cooperation among stakeholders.

Risk calculation can, in some embodiments, be determined through the evaluation of the payoff matrix for the set of games representing the interactions of a set of stakeholders, in that if there is a predominant strategy, such a strategy can be evaluated in terms of the incentives of the stakeholders to identify values for those incentives that are most likely to cause misalignment.

The risk can be determined, in part through evaluation of the incentive values in relation to the payoff matrix, which can inform the decisions of the stakeholders. Such risk calculations can be expressed from the perspective of the system, one or more stakeholders, devices, sensors or other system entities. In this manner such risk calculations may, in turn, form the basis for a further game, where the payoff matrix may be used as a basis for a decision matrix, in whole or in part, enabling a risk management strategy that mitigates incentive misalignment.

In some circumstances, should a misalignment of incentives be identified, for example using predictive digital twins, such as Care Village Digital Twins (CVDT's), a message may be sent to at least one stakeholder involved in such a misalignment to alert them to this situation. In this manner such a message may encourage a stakeholder to adapt their behaviors so as to mitigate or remove the misalignment and/or encourage the involved stakeholders to revise their actions in light of the communications.

A pattern may be represented by a game, including multiple strategies in a game representing a pattern, and then “distance” of measurements from boundaries indicates direction (vectors) of trajectory from compliance to non-compliance

One aspect of the machine learning systems is the determination of the rate of change of at least one data set, such that the for example if a pattern is operating that pattern represents the initialization of the data set that is to be used as the corpus for the application of the machine learning techniques. For example, an operating CVDT may have a set of data (X) provided by the devices, sensors, care processing and/or other care village systems. The machine leaning may use various techniques, including inference, deep learning and various instantiations of Boltzmann machines to determine, at least in part, a maximum for a that data set in part or in whole on one or more axis, variables or other portion of the data. Such predictive operations may then be used, at least in part, to configure one or more sensors, devices and/or systems and/or other data providing and/or processing systems. These operations may also be used to establish an end point for a set of interactions, such as that represented in a decision matrix produced as a result of one or more games, such that the incentives and their values, the strategies deployed within the game and end payoffs forming such decision matrix may be evaluated to determine any misalignments of these incentives and values, that are indicative of at least one stakeholder attempting to rort the care village systems and/or stakeholders, or disadvantage another stakeholder in a manner contrary to the operations of the care village. As it is impractical to try and define in a classic rule set approach, the potential interactions of stakeholders, especially in situations with more than two stakeholders are involved, the use of game theory and machine learning enables the capability of identification of potential and actualized incentive misalignments, which can inform and/or configure at least one care village response system before, during or after the time of such a misalignment, so as to prevent, reduce, mitigate or correct such misalignment.

In some embodiments, adaptive (including dynamically adjusting) boundary conditions and/or constraints sets may be deployed, where such conditions and constraints have been identified, at least in part by machine learning techniques. This can include identification of causal possibilities/probabilities, in terms of relationships between and amongst, devices, sensors, systems, stakeholders, games and the data sets they produce. This can include identification of new variables, weightings for those variables, knowledge-based conditions, context variations, matching and the like. Part of the benefit of this approach is the mitigation of unforeseen use cases including those based on incentive alignments and weightings.

One aspect of the care village machine learning systems deployments can involve generative AI for enhanced messaging for one or more actions.

In some embodiments, an automated system can be entrusted with the task of remedying, mitigating, or even preventing identified incentive misalignments or their potential risks. This system can employ a range of strategies, including communication with the relevant stakeholders. The objective of these interactions is to engender changes in stakeholders' behavior that align with desired outcomes. The communication can take various forms, ranging from one-way communications to interactive dialogues using one or more communication channels.

To optimize these communication efforts, generative AI models can come into play. These models generate content that is tailored to the specific context and stakeholders involved. By analyzing historical communication patterns, stakeholder preferences, and the desired objectives, the generative AI can produce nuanced, informative and/or persuasive messages. These messages are strategically designed to resonate with stakeholders, encourage cooperation, and foster a shared understanding of the importance of alignment, and may focus on the common good and/or in the stakeholder's own interests.

Compliance

One aspect of the system is the ability to evaluate and/or track compliance of a stakeholder in relation to specifications that represent that compliance. For example, if a stakeholder, such as a carer, is contracted to attend a PUM at a location within a specified time window, then one or more sensor, device and/or system can be instructed to provide data within that time window, that can confirm or not the attendance of the contracted stakeholder. This approach can involve “hard” data, such as proximity connections, where for example the PUM has a device and the carer has a device that when in proximity create an event data stream that confirms the contracted period. Further sensors, such as entry/exit sensors, cameras, motion detectors and the like can then provide further data sets that can be matched to the pattern of activity that represents the behaviors and interactions of both PUM and carer. In this example each of the PUM and the carer may have an individual pattern and together may have a common or shared pattern. Such patterns may be represented by one or more tokens. In this manner the degree of deviation from any specified event or occurrence may be considered within the context of the interactions of these patterns and as such represents a significantly more flexible and adaptive monitoring system when compared to rules-based systems. This monitoring may be undertaken with digital twins including sets thereof, where for example the environment, represented by a digital twin and the stakeholders, both PUM and carer are also represented by digital twins, such that there is a combination of the digital twins where the data from sensors of the environment, PUM and carer are combined, as are the patterns, to create a unified representation of this interaction. Such an interaction may be, for example, be hosted in a manner where a monitoring function oversees the combined pattern sets. Such a monitoring system or module may be configured to create alerts or events in accordance with compliance specifications, such that if data does not match the expected, configured and/or specified compliance parameters, an exception may be generated.

These exceptions may be passed to an exception handling system which can then undertake the appropriate remedial actions, if required and/or provide an appropriate report to the relevant systems, persons and/or other controlling entities.

The identification and detection of activity that has a deviation from agreed terms, specifications and/or behavior norms can be beneficial in tracking and managing the changes in circumstances, for example caused by a variance in the health condition of a PUM, and/or the more gradual changes in behaviors of a set of stakeholders over time. In this manner the gradual variations that tend to occur as stakeholders interact and the relative variations on the incentives of and for those stakeholders may be identified and where appropriate a response may be initiated. This can lead to the deployment, on a graduated and timed basis, of corrective responses, for example the variation of an incentive for a stakeholder. For example, a change in the time of an appointment, increase in a payment, reduction in workflow or other benefit or incentives can impact the overall balance of incentives of a set of stakeholders before a more significant impact, outcome or behavior change, including where that behavior change includes rorting the system, may be avoided. This tendency for a minor deviations and corrections to occur in any set of interactions can be gradual, often over multiple iterations and as such any responses and/or corrective actions may be similarly undertaken in a gradual series of iterations.

In some embodiments, specifications for compliance may be determined and expressed in terms of alignment of incentives, where for example the care village systems may present a set of specifications that represent the incentives for each of the stakeholders from the perspective of the care village. Each of the stakeholders may also express their own incentives and represent these as part of their specifications that, at least in part, are represented by their digital twin. Although the specifications of the incentives of each party may represent, for example differing and potentially contradictory values as expressed by those stakeholders, these specifications can form the basis for compliance of each of the parties as they interact.

Such compliance can be evaluated and, for example using a matching system to establish the compliance of such specifications with events, including sequences thereof, and any actions.

One aspect of incentive analytics operations is the capability, through for example environment sensing management systems and/or care processing systems to configure a sensor, device and/or system or set thereof, to provide additional data on an occurrence or sequence of occurrences so as to provide more detailed, focused, enhanced and/or additional sensing capabilities. Such an approach can be used to determine whether an occurrence is a single event within a context, such as forgetfulness, response to a stakeholder request or other minor variation or an attempt by a stakeholder to rort or manipulate a situation in a manner that is beneficial to them and detrimental to another stakeholder and/or the care village systems.

The enhanced and/or focused sensor, device and/or system data may form, in part, the determining factor in an evaluation of a responses to an occurrence. For example, if a sensor, device and/or system captures an audio signal that could be the result of a PUM falling, then a camera sensor may be enabled to verify that data set, as may other sensors, such as motion detectors and the like, so as to create a more comprehensive data set representation of the situation. If for example, a carer was in the same room as the PUM, and the fall is verified, then the actions of the carer may be considered, especially if they are close to the PUM at the time of the fall. As in many cases a camera can be capturing the images, though not transmitting those images to any other systems, the system may be able to replay the events to determine whether the carer was instrumental in the fall or had acted to prevent or mitigate the fall.

It can be clearly understood that this approach could be both computationally and administratively overbearing and overwhelming, however the use of multiple levels of configuration and pattern, for example represented by tokens, deployment with in a known context, such as a HCP within the care village enables the care village systems to effectively evaluate any variations and deviations, so as to separate different strategies employed by a stakeholder in the legitimate execution of their duties or actions and those which represent an attempt to exploit a situation in a manner that is malicious and/or detrimental to another stakeholder and/or the care village.

Stakeholder interactions may be represented by games that can be evaluated using game theory. Within these games there may be strategies that for the majority of the interactions are accurate representations of the intentions of the stakeholders, and as such can be used to determine the compliance of the stakeholders with the specifications, patterns or other care village systems. This can include, for example the determination, by specification and/or evaluation of the common strategies that can be employed by the stakeholders within a game. For example, this can include the preferred strategy, that is the one that as determined by the care village systems, is the strategy that achieves an outcome that is most beneficial to both stakeholders. For example, a carer may visit a PUM to administer a medicine, and stay with the PUM for a period to ensure that there are no untoward after effects of the medicine. The incentives for the carer to cut short that visit may be influenced by the number of other visits they have scheduled and the incentive for the PUM may be to extend the time of the visit as they seek company. There may be a number of strategies employed in the game representing this interaction, of which one for each of the stakeholder represents an equilibrium. Such strategies may then be preferred and any variations from these strategies can be considered as deviations, to a greater opt lesser degree from compliance with the preferred outcome.

Such games may form part of a pattern, in that the pattern may represent the sensed behaviors of the stakeholders, and the game may represent the interactions of those stakeholders. The combination of sensor-based data for early detection/reaction/monitoring of the events unfolding and the variations, if any, form the preferred strategies, can provide detections of variations in the patterns and the variables thereof. These variations can be evaluated in terms of compliance with specifications that may govern these interactions as well as providing data as to changes in the state of the stakeholders, including the PUM and their wellness condition.

Patterns and their context in HCP provide continuously variable detection capabilities with real time/near real time responses. The HCP provides a structure to the likely health journey of a PUM, based on the initial diagnosed health and/or wellness condition that initiated this monitoring. This structure, over time, experience, health conditions and environment can be represented by a set of patterns, which can be expressed as tokens, which are, at least in part, the representation of the state of the PUM in an environment. The HCP, at least in part, provides the context for many of the states, patterns, tokens and the data represented by them, and as such can effectively provide a context for any incentive misalignments, particularly those that are formed, at least in part, by the operations of one or more stakeholders. For example, if a stakeholder has a set of policies that are, in whole or in part, in contradiction to or orthogonal to, the interests and incentives of another stakeholder, then there is a high probability of incentive misalignment which can lead to behaviors to mitigate, negate or otherwise impact such policies. The FAPS system, employing game management systems, machine leaning/artificial intelligence, CVDT and other care village systems may operate to minimize such misalignments.

In some embodiments, the measurement of deviations from the pattern that is operating may indicate a trend, which when projected, for example using one or more CVDT, can indicate that the equilibrium of the incentives within a game representing interactions between stakeholders is varying, such as decaying. This can inform the care village systems and the stakeholders involved of such a trend so as to produce a configuration that can be passed to the stakeholder, devices, sensors and/or systems. This can be in the form of compliance-based specifications and/or instructions where transactional or other consequences may be involved. For example, if a carer extends their lateness for an appointment over a series of visits to a PUM, or makes each visit shorter, then for example, timing and/or duration of the visit may be varied.

The responses to variations in compliance or noncompliance may be, in some embodiments, executed in a rigid or flexible manner. A rigid response may be, for example threshold based, in that if a service is not provided there is no payment for that service. This is typical of rules-based systems and often leads to inflexible outcomes that do not meet the needs of the stakeholders involved, and in some circumstances can cause hardship, distress and/or other health and wellness impactful negative outcomes.

A further response may be a flexible response, where the incentives of the stakeholders within the scope of the interactions are evaluated to formulate one or more variations of those incentives that brings into equilibrium the respective stakeholder incentives such that neither stakeholder is wholly disadvantaged and the impact and/or any disadvantage of a stakeholder from the perspective of their wellness, financial or other impacts is minimized and/or mitigated. For example, varying an appointment time so that a carer may take their child to school, or selecting a carer with whom a stakeholder has a positive reaction and the like. This may in some embodiments be achieved though compliance specifications incorporating one or more degrees of flexibility and incorporating the incentives and their values as part of such compliance specifications.

The data sets provided by the devices, sensors and/or systems in collaboration with the patterns, operating and predictive support the use of adaptive variations in compliance criteria, expressed as specifications, caused for example, by context change. For example, when the monitoring systems predict changes of patterns representing PUM wellness state changes, which may for example, be represented by one or more tokens, this may include the generation and/or propagation of alerts/events for support of the transition and/or new pattern, which in turn may influence contextual variations including, for example, scheduling, supply chain, stakeholder choices and the like. In this example compliance specifications can be adjusted in response to these changes, either automatically and/or with human and/or machine learning intervention.

Outcomes and Response Systems

The care village can incorporate one or more response systems, which can be configured to undertake responses to situations that are determined to be in need of a response from the care village. These responses can be automatic and/or manual and can involve care village systems and/or external systems, including emergency services, in any arrangement.

The response systems may be configured so as to interact with, for example, the environment sensing, care processing, token systems, incentive misalignment, devices, sensors, systems, stakeholders, external to the care village systems and/or any other entity or party as specified in any arrangement.

The response systems interacting with care village systems may employ encrypted and other tokens, encrypted messages, secure communications and/or messaging systems configured for the operations within the care village. This can include the use of API's and authentication, authorization and/or access controls so as to ensure the security, privacy and/or legitimacy of the communications, which includes any privacy and confidentiality standards deemed appropriate for this type of messaging and communications.

In some embodiments, these messages and communications may, in whole or in part, may be recorded in at least one distributed ledger. In some cases, these communications may form part of a smart contract. Such communications may employ on or more tokens.

One aspect of the care village systems is the instantiation of responses to various situations, where the response time is essential to the wellness state of at one or more stakeholder, such as the PUM. In this example, the response systems include a prioritization capability that may, for example, use multiple communication methods to communicate a message, including obtaining an acknowledgement, to one or more stakeholder and/or external service, for example an emergency service. The care village is not intended to supplant a sovereign, state, county or city mandated emergency system, rather the intention is for the care village to provide communications to those systems when appropriate. The response systems may enable, for example, certain access to sensors, devices and/or other care village systems to such emergency services for the period when they are activated and operational in regard of one or more stakeholder. The selection and use of the data so provided may be subject to pre agreed arrangements with such services, so as to enable those services to operate in a manner beneficial to the stakeholder whose wellness is in question.

The use of game theory within the system, coupled with machine learning enables the embodiment of at least one decision matrix incorporating multiple decision combinations, for example game payoffs, alternatives based on differing strategies, temporal states, differing inputs and the like.

In some embodiments, based on the types of game deployed and the number of stakeholders involved in the game, there may be a set of payoff matrix that can be represented in the form of at least one decision matrix. Such a decision matrix can, in some embodiments, form part of a decision engine module which incorporates one or more weightings, parameters, attributes or other characteristics that, in whole or in part, may be used to and/or inform risk evaluation systems, such as for example risk assessment modules. In this manner potential responses may be considered for their impact, particularly in relation to influencing one or more stakeholders so as to potentially vary their actions in light of their stated or intended incentives, so as to mitigate and/or negate any incentive misalignments.

In some embodiments, the resulting decision matrix may be represented by a lattice, whereby the nodes and edges of the lattice incorporate the decisions and their relationships to the stakeholders, their incentives for that game, their strategies, including deployed, potential and respective payoffs. This decision matrix may be used as a corpus for machine learning, for example to determine feature sets and/or may be the result of machine learning applied to the interactions of the stakeholders, including as represented by a CVDT of their current state and potential CVDT's that anticipate their one or more potential states. The use of multiple game types using differing or same inputs, with the potential strategies evaluated from the perspective of the incentives of the stakeholders provides a data set that when expressed as a decision matrix, can be used to determine the set of most likely outcomes of those interactions and as such can then be used to configure one or more system, device and/or sensor in anticipation of and/or in response to one or more wellness event.

In some embodiments such decision matrix may be created in anticipation of actual or forthcoming events and as such, comparisons between the actual state as represented by the stakeholder interactions, devices, sensors and systems may be evaluated in relation to those decision matrixes to identify whether these outcomes match specified, predicted and/or declared outcomes.

The determination of any variations from the predicted decision matrix and the actual decisions can then be issued to formulate further game theory representations of those interactions, evaluate incentives and their values in light of those decisions, identify other incentives that may be pertinent to those interactions and the like.

This approach when combined with the use of HCP and patterns thereof, provides for those patterns to more accurately represent the likely and actual interactions between stakeholders within the care village. One aspect of this is the identification and representation of new patterns that are sufficiently divergent from those being operational, and as such may represent a variation in the incentives and values thereof, the wellness of a PUM, a change in circumstances of a stakeholder or other factors that have resulted in such a new pattern being identified.

Combinations, Cooperation, Collaboration and Collusion

One aspect of the system is the determination, through real time and/or predictive monitoring, of a sequence of events within and across multiple patterns, which may be represented as tokens, where such events can involve multiple parties. For example, the set of stakeholders involved in a particular situation, may include the PUM, a carer, a medical professional and an insurance organization. In this example, there may be multiple games that represent the interactions between the parties, for example there may be a hierarchy of games which can include various cooperative strategies being employed. For example, the financial cost of a service provision is likely borne by the insurance organization and as such, a combination of PUM and carer who cooperate, may result in the payment for a service that was not delivered. Additionally, the cooperation of a medical professional with a service provider, for example a carer, may result in billing the insurance organization for services that are either not delivered or are not required.

The use of patterns, representing the actual and predicted behaviors of the stakeholders individually and collectively, combined with the representation of their interactions in the form of games, resulting in payoff matrix that represents one or more decision matrix can provide a unique insight, when analyzed through the lens of incentives and values thereof, as to the intentions, legitimacy and/or compliance of such stakeholders within the care village.

The evaluation of the data provided by the devices, sensors and/or systems that form, at least in part, patterns, both operating and predicted, in combination with outcomes as represented by payoffs from games in the form of decision matrix provide a unique approach to detecting and identifying data patterns that span, the sensing, interactions and outcomes of stakeholder interactions, based at least in part on repeated occurrences of such data. One aspect of this is determining the variations from existing operating patterns, where such a variation may be minor, however the repeated nature of the data forming such variation can inform one or more systems and/or humans involved in the monitoring, of the pattern created by these variations. When compared with the incentives and their values, these data can be used to deploy at least one response, so as to identify, correct and/or mitigate any incentive misalignments. In some embodiments, this may comprise a new pattern that may be an overlay on one or more existing patterns.

For example, such variation may be in the timekeeping of a stakeholder, for example they keep increasing (3 min late/5 min late/10 min etc.) their lateness. A pattern could change permanently or periodically, for example seasonal patterns, recurring health condition patterns and the like, and such variations may be indicators of such changes.

In some embodiments, one or more CVDT may be used, to at least in part, determine the set of predicted interactions based, at least in part, on the games employed and the players involved. Such an approach can involve creating a non-deterministic predictive interactive systematic model, which at least in part, can represent the one or more incentives of the players involved, expressed through their strategies in the one or more games.

In some embodiments, interactions between stakeholders where the incentives of those stakeholders' influence, in whole or in part those interactions, may be represented by nodes, for example as nodes in a graph database. These nodes may, for example comprise a set of interactions, expressed as a sequence of events or actions of each of the stakeholders, where for example each of these actions is a move in a game in which each of the stakeholders is a player. These interaction nodes may form sets, which are processes that are undertaken, potentially in an automated manner, that represent a sequence of interactions expressed, for example as a pattern. In this manner, each of the stakeholders' incentives, represented by their actions may be played within a game to achieve an outcome of that game represented by the payoffs of that game. In some embodiments, events detected by a sensor may result in those events forming sequences, where each sequence, may for example, traverse a set of nodal interactions, as represented for example in a graph database, as for example a transaction unfolds.

Smart Contracts and Patterns

Smart contracts (SC) refer to computer protocols described as computer code that runs in the protected computing environment of a Distributed Ledger (DL) virtual machine, such as for example, the Ethereum VM, and that digitally facilitate the verification, control, and/or execution of transactions, according to pre-established and immutable agreements and rules. SC behavior cannot be modified and the data resulting from their execution can be directly and securely stored in the DL, which makes it impossible or very difficult to perform any external manipulation.

In some embodiments a DL storage can be combined with one or more SC in such a way that an activity, such as a transaction, is only validated and/or can continue once a related SC is executed, and that execution involves storing data of that activity in a DL. As a result, an immutable trace of key stakeholder activities is kept in the DL, which can be used to verify proper patterns and/or sequences, and to identify non-proper patterns and/or sequences. In some embodiments this SC and DL tracking mechanism can be applied to real-life activities, in others it can use DT simulated activities and, in others, a combination of real-life and DT simulated activities.

Some embodiments can use the SC and DL tracking mechanism to store patterns identified, predictions generated, decisions made from those, and execution activities based on those decisions, resulting in an immutable trace of the misalignment avoidance system workings, which can be used for reporting purposes, auditing, forensics and the like.

In some embodiments one or more SC can be used in such a way that an activity, for example a transaction, is only validated if the one or more related SC are executed. This SC set can take inputs form the activity, from real-life signals/inputs or patterns, Digital Twins, for example CVDT, simulated signals/inputs or patterns, or from a combination of real-life and simulated ones. They can also use data stored in a DL and/or a traditional storage method, as well as signals and data from external inputs. The SC can apply conditions, make computations and generate a result that validates or invalidates the activity. The SC may also include the mechanism that automatically executes an operation as a result from the inputs, such as activating a payment, generating a positive or negative review or score, notifying the involved parties, triggering the start of another activity, etc.

Tracking and validation using SC and DL can be done at different levels of granularity within the Care Village activities. For example, a generic/high-level SC set can be used to validate high-level activities, without looking into details or sub-activities within those high-level activities. If a particular pattern is identified in one of those high-level activities, and that pattern is associated with higher risk of incentive misalignment, a second SC set can be activated to validate sub-activities which are part of the high-level activity that presents the higher risk. This dynamic change of granularity for validation and tracking using SC and DL can be applied to real-life, simulated or combined scenarios. Different levels of risk, different incentive misalignments and other conditions may require different methods for validation and/or tracking, therefore there may exist different SC sets covering the needs of such different conditions and a combination of selection and/or decision systems may be used to select the appropriate SC set to use in a particular case.

In some embodiments the decisions of whether a high-level activity presents higher risk of incentive misalignment, whether to activate lower-level tracking and validation based on SC and which set of SC to activate can be made based on a ML enabled system. In such cases a ML enabled system, such as a neural network, can be trained and used to take inputs related to a high-level activity and produce an output that associated the inputs with a known pattern, using segmentation, classification, detection and/or other methods. This output can be used by a decision subsystem, based, for example, on a decision matrix, to determine if any sub-activities of the high-level activity need to be lower-level verified and/or tracked using SC and DL and which set of SC are the most appropriate for such verification and/or tracking. Some embodiments may apply game theory games and elements thereof, such as payoff matrix and/or game trees, to select the need for SC and DL base validation and/or tracking and which SC set to use.

Smart contracts may be held in a repository as may pattern specifications, and segments thereof, where by each of SC may have a relationship with a pattern, or segment thereof. This capability to integrate patterns and SC, such that the process of creation of SC may be automated at the appropriate level of granularity is particularly useful in the cases where the PUM is in a quiescent state, such that the state is used as a formalism, for example, as a behavior token (Bevoken), in regard of the events and event sequences that are undertaken and as such provide the capability to write such event occurrences in an immutable manner to a distributed ledger.

Although an SC cannot be amended, they can be replaced and in the case of patterns, there may be multiple SC, or portions thereof that have relationships with the patterns, or segments thereof, such that through replacement, extension, addition and/or accumulation the SC and records in the DL may accurately represent the events.

In some embodiments, the SC and pattern alignment can be configured for appropriate granularity to meet the degree of flexibility required in proving an audit trail for an HCP and the patterns therein. For example, an SC may be derived or extracted for a pattern, including segments thereof, such that when the data sets have satisfied the pattern configurations the SC is generated and the results written to the appropriate DL. This can include the use of one or more systems that ensure the accuracy and provenance of the patterns, data sets, SC relationships and any conditions and their satisfaction within these entities.

In some embodiments, a DL may be used as a record of training, either of human stakeholders and/or of machine learning systems. For example, if a corpus of material is evaluated by one or more machine learning systems, for example to determine, at least in part, unstated incentives of a set of stakeholders, where the data under evaluation is a record of their interactions, this training may be represented as quantized outcomes in one or more distributed ledgers. The same approach can be undertaken for a human stakeholder, such as when a person is assisting a qualified carer to learn the appropriate skills. Such records may include or exclude various stakeholders, for example through the use of tokens so as to anonymize the identity of one or more stakeholder.

Game strategies can be instantiated in smart contracts as may be game payoffs/decision matrix and the like, creating an immutable record of the execution by one or more game and/or the results thereof. For example, select from game set A, execute games A1, A2 and A3 in a sequence.

In some embodiments at least one protected processing environment (PPE) may be employed in lieu of, in support of and/or in conjunction with at least one smart contract and/or distributed ledger. For example, a protected processing environment may be used to process incentive specifications and their values in relation to stakeholder interactions. The outcome of those interactions may be processed by a PPE and written directly or indirectly to at least one digital ledger. For example, one or more tokens may be employed in this manner.

The previous description of the embodiments is provided to enable any person skilled in the art to practice the disclosure. Thus, the present disclosure is not intended to be limited to the embodiments shown herein, but is to be accorded the widest scope consistent with the principles and features disclosed herein.

What is claimed is:

1. A system to align tokens representing the state of a person under care with at least one stakeholder represented by further tokens, comprising:
a plurality of environmental sensors configured to monitor a sequence of interactions of a person under care resulting in a detected data set, and to provide a first token, the first token comprising the detected data set representing quiescent behaviors of the at least one stakeholder in an environment;
each of the behaviors is represented by a multi-dimensional feature set comprising at least one pattern forming part of a health care profile for the person under care;
a fraud avoidance prediction system comprising:
a transceiver configured to receive the first token from the plurality of environmental sensors;
a non-transitory computer-readable storage medium configured to store a second token, the second token representing current state behaviors of the at least one stakeholder in the environment, and
at least one hardware processing unit to compare the first token and the second token, and use at least in part game theory to align the first token and the second token.

2. The system of claim 1, wherein the comparison of the first token and the second token results in at least one notification to the at least one stakeholder.

3. The system of claim 2, wherein the alignment of the first token and the second token is via a consequent incentive for the at least one stakeholder.

4. The system of claim 2, wherein the alignment of the first token and the second token aligns configurations of the plurality of environmental sensors.

5. The system of claim 4, wherein the game theory is a cooperative game, a normal form or extensible form game.

6. The system of claim 4, wherein the game theory is a simultaneous or sequential move game.

7. The system of claim 4, wherein the game theory is a constant sum, zero sum, non-zero-sum game, symmetric or asymmetric game.

8. A machine-learning enabled fraud-detection system to align tokens representing the state of a person under care with at least one stakeholder represented by further tokens, comprising:
a transceiver configured to receive a detected data set from a plurality of environmental sensors, the detected data set representing quiescent behaviors of the at least one stakeholder in an environment;
each of the behaviors is represented by a multi-dimensional feature set forming part of a health care profile for the person under care;
at least one machine-learning processing unit configured to receive the detected data set from the transceiver and to create a first token comprising the detected data set;
a non-transitory computer-readable storage medium configured to store a second token, the second token representing previous representing current state behaviors of the at least one stakeholder in the environment, and
the at least one hardware processing unit further configured to compare the first token and the second token, and use at least in part game theory to align the first token and the second token.

9. The system of claim 8, wherein the comparison of the first token and the second token results in at least one notification to the at least one stakeholder.

10. The system of claim 9, wherein the alignment of the first token and the second token is via a consequent incentive for the at least one stakeholder.

11. The system of claim 9, wherein the alignment of the first token and the second token aligns configurations of the plurality of environmental sensors.

12. The system of claim 11, wherein the game theory is a cooperative game, a normal form or extensible form game.

13. The system of claim 11, wherein the game theory is a simultaneous or sequential move game.

14. The system of claim 11, wherein the game theory is a constant sum, zero sum, non-zero-sum game, symmetric or asymmetric game.

15. A non-transitory computer-readable storage medium encoded with data and instructions, the instructions when read by a computer causes the computer to:
receive, via a transceiver, a detected data set from a plurality of environmental sensors, the detected data set representing quiescent behaviors of the at least one stakeholder in an environment;
each of the behaviors is represented by a multi-dimensional feature set forming part of a health care profile for the person under care;
receive, via at least one hardware processing unit, the detected data set from the transceiver and to create a first token comprising the detected data set;
store, via the non-transitory computer-readable storage medium, a second token, the second token representing current state behaviors of the at least one stakeholder in the environment, and compare, via the at least one hardware processing unit, the first token and the second token, and use at least in part game theory to align the first token and the second token.

16. The non-transitory computer-readable storage medium of claim 15, wherein the comparison of the first token and the second token results in at least one notification to the at least one stakeholder.

17. The non-transitory computer-readable storage medium of claim 16, wherein the alignment of the first token and the second token is via a consequent incentive for the at least one stakeholder.

18. The non-transitory computer-readable storage medium of claim 16, wherein the alignment of the first token and the second token aligns configurations of the plurality of environmental sensors.

19. The non-transitory computer-readable storage medium of claim 18, wherein the game theory is a cooperative game, a normal form or extensible form game.

20. The non-transitory computer-readable storage medium of claim 18, wherein the game theory is a simultaneous or sequential move game.
